(19) United States
(12) Patent Application Publication
(10) Pub. No.: US 2008/0069870 A1
(43) Pub. Date: Mar. 20, 2008

(54) Controlled Release Compositions Comprising a Cephalosporin for the Treatment of a Bacterial Infection

(75) Inventors: Scott A. Jenkins, Downingtown, PA (US); Gary Liversidge, Westchester, PA (US)

Correspondence Address: SYNNESTVEDT & LECHNER LLP, 2600 ARAMARK TOWER, 1101 MARKET STREET, PHILADELPHIA, PA, 19107, US

(73) Assignee: Elan Corporation Pic, Dublin 2 (IL)

(21) Appl. No.: 11/571,379

(22) PCT Filed: Apr. 12, 2006
(86) PCT No.: PCT/US06/13637

§ 371 Date: Nov. 8, 2007

Related U.S. Application Data
(60) Provisional application No. 60/670,417, filed on Apr. 12, 2005.

(51) Int. Cl. A61K 31/546; A61K 9/10; A61K 9/14; A61K 9/24; A61K 9/58; A61P 31/04

(52) U.S. Cl. 424/456; 424/472; 424/484; 424/490; 514/204

(57) ABSTRACT

The invention relates to a controlled release composition comprising a cephalosporin that in operation delivers the drug in a pulsed or bimodal manner for the treatment of bacterial infection. The controlled release composition comprises an immediate release component and a modified release component; the immediate release component comprising a first population of cephalosporin-containing particles and the modified release component comprising a second population of cephalosporin-containing particles coated with a controlled release coating; wherein the combination of the immediate release and modified release components in operation deliver the active ingredient in a pulsed or bi-modal manner. Preferably, the cephalosporin is cefcapene pivoxil or a salt thereof which can be released from the dosage form in an erodable, diffusion and/or osmotic-controlled release profile.

### FIELD OF INVENTION

The present invention relates to a novel method for treating patients suffering from a bacterial infection. In particular, the present invention relates to a novel dosage form for the controlled delivery of a cephalosporin, such as cefcapene pivoxil or a salt thereof.

### BACKGROUND OF INVENTION

Antibiotics are powerful bacteria-killing drugs used to treat bacterial infection in humans and other mammals. There are hundreds of antibiotics currently in use, most tailored to treat a specific kind of bacterial infection. Beta-lactam antibiotics, which are named for the beta-lactam ring in their chemical structure, include the penicillins, cephalosporins and related compounds. These agents are active against many gram-positive, gram-negative and anaerobic organisms. The beta-lactam antibiotics exert their effect by interfering with the structural crosslinking of peptidoglycans in bacterial cell walls. Because many of these drugs are well absorbed after oral administration, they are clinically useful in the outpatient setting.

The cephalosporin beta-lactam antibiotics are a group of semi-synthetic derivatives of cephalosporin C, an antimicrobial agent of fungal origin. They are structurally and pharmacologically related to the penicillins. The cephalosporin ring structure is derived from 7-aminocephalosporanic acid (7-ACA) while the penicillins are derived from 6-aminopenicillanic acid (6-APA). Both structures contain the basic beta-lactam ring but the cephalosporin structure allows for more gram negative activity than the penicillins and aminocillins. Substitution of different side chains on the cephalosporin ring allows for variation in the spectrum of activity and duration of action.

Cephalosporins are grouped into “generations” by their antimicrobial properties. The first cephalosporins were designated first generation while later, more extended spectrum cephalosporins were classified as second generation cephalosporins. Currently, three generations of cephalosporins are recognized and a fourth has been proposed. Significantly, each newer generation of cephalosporins has greater gram negative antimicrobial properties than the preceding generation. Conversely, the “older” generations of cephalosporins have greater gram positive coverage than the “newer” generations.

Cephalosporins are used to treat infections in many different parts of the body. They are sometimes given with other antibiotics. Some cephalosporins given by injection are also used to prevent infections before, during, and after surgery.

Like other cephalosporins, cefcapene is a cephalosporin which demonstrates its bacterial activity by inhibiting synthesis of the bacterial cell wall. Cefcapene exhibits a broad spectrum of antibacterial activities in vitro against microorganisms ranging from aerobic and anaerobic gram-positive and gram-negative bacteria. Cefcapene also exerts antibacterial activity against penicillin-resistant Streptococcus pneumonia and ampicillin-resistant Haemophilis influezae.

Cefcapene pivoxil hydrochloride, abbreviated CFPN-PI, is offered under the registered trademark FLOMOX® by Shionogi & Co., Ltd. of Japan. CFPN-PI has the chemical name 2,2-Dimethylpropanoyloxymethyl (6R, 7R)-7-[(Z)-2-(2-aminothiazol-4-yl)pent-2-enylamino]-3-carabmoyloxymethyl-8-oxo-5-thia-1-azabicyclo[4.2.0]oct-2-ene-2-carboxylate monohydrochoride monohydrate. CFPN-PI has the molecular formula C₂₃H₂₉N₅O₈S₂ HCl H₂O with a molecular weight of 622.11. The structural formula of CFPN-PI is:

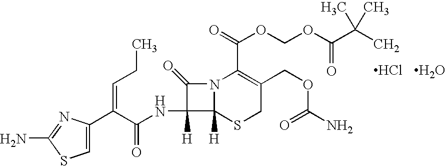

CFPN-PI is a white to pale yellowish-white, crystalline powder or mass. It has a faint, characteristic odor, and has a bitter taste. It is freely soluble in N, N-dimethylformamide and methanol, sparingly soluble in ethanol only slightly soluble in water, and practically insoluble in diethyl ether.

A typical adult dosage of CFPN-PI is about 100-150 mg administered orally as 75 mg or 100 mg tablets three times daily after meals. The absorption of CFPN-PI is known to be better after meals than before meals. CFPN-PI is hydrolysed into its active metabolite, cefcapene, upon absorption by esterase in the intestinal wall.

Cefcapene pivoxil is used to treat conditions including, but not limited to, superficial skin infection, deep skin infection, lymphangitis, chronic pyoderma, secondary infections in trauma, burns, and surgical wounds, mastitis, periproctic abscess, pharyngolaryngitis, tonisilitis, acute bronchitis, pneumonia, secondary infections in chronic respiratory diseases, cytstitis, pyelonephritis, urethritis, cervicitis, cholecystitis, cholangitis, bartholinititis, intrauterine infection, uterine adnexitis, dacryocyctitis, hordeolum, tarsadenitis, otitis externa, otitis media, sinusitis, periodontal tissue inflammation, pericoronitis, and gnathitis. Bacterial strains known to be susceptible to cefcapene pivoxil include, but are not limited to, Staphylococcus sp., Streptococcus sp., Pneumococcus sp., Neisseria gonorrhoeae, Moraxella (Branahainela) catarrhalis, Escherichia coli, Citrobacter sp., Klebsiella sp., Eneterobacter sp., Seriatia sp., Porteus sp., Morganella inorganii, Providencia sp., Haemophilis influenzae, Peptostreptococcus sp., Bacteroides sp., Prevotella sp. (excluding Prevotella bivia), and Propionibacterium acnes.

Cephalosporins such as cefcapene pivoxil are of high therapeutic value for the treatment of bacterial infections. Given that cephalosporins such as cefcapene pivoxil require oral administration three times daily, strict patient compliance is a critical factor in the efficacy of cephalopsorins in treating bacterial infections. Moreover, such frequent administration often requires the attention of health care workers and contributes to the high cost associated with treatments involving cephalosporins such as cefcapene pivoxil. Thus, there is a need in the art for cephalosporin compositions which overcome these and other problems associated with the use of cephalosporins for the treatment of bacterial infections.

The present invention then, relates to a composition for the controlled release of cephalosporins. In particular, the present invention relates to a composition that in operation delivers an active cephalosporin, such as cefcapene pivoxil or salts thereof, in a pulsatile or in a constant zero order release manner. The present invention further relates to solid oral dosage forms containing such a controlled release composition.

### DESCRIPTION OF THE INVENTION

The plasma profile associated with the administration of a drug compound may be described as a “pulsatile profile” in which pulses of high cephalosporin concentration, interspersed with low concentration troughs, are observed. A pulsatile profile containing two peaks may be described as “bimodal”. Similarly, a composition or a dosage form which produces such a profile upon administration may be said to exhibit “pulsed release” of the cephalosporin.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash out period provided by the fall off of the plasma concentration of the active between peaks has been thought to be a contributing factor in reducing or preventing patient tolerance to various types of drugs.

Multiparticulate modified controlled release compositions similar to those disclosed herein are disclosed and claimed in the U.S. Pat. Nos. 6,228,398 and 6,730,325 to Devane et al. both of which are incorporated by reference herein. All of the relevant prior art in this field may also be found therein.

Accordingly, it is an object of the present invention to provide a multiparticulate modified release composition containing a cephalosporin, preferably cefcapene pivoxil or a salt thereof, which in operation produces a plasma profile substantially similar to the plasma profile produced by the administration of two or more IR dosage forms given sequentially.

It is a further object of the invention to provide a multiparticulate modified release composition which in operation delivers a cephalosporin, preferably cefcapene pivoxil or a salt thereof, in a pulsatile manner.

Another object of the invention is to provide a multiparticulate modified release composition which substantially mimics the pharmacological and therapeutic effects produced by the administration of two or more IR dosage forms given sequentially.

Another object of the present invention is to provide a multiparticulate modified release composition which substantially reduces or eliminates the development of patient tolerance to a cephalosporin, preferably cefcapene pivoxil or a salt thereof, of the composition.

Another object of the invention is to provide a multiparticulate modified release composition in which a first portion of a cephalosporin is released immediately upon administration and a second portion of the active ingredient is released rapidly after an initial delay period in a bimodal manner.

Another object of the present invention is to formulate the dosage forms as erodable formulations, diffusion controlled formulations, and osmotic controlled formulations and deliver the drug in a zero order fashion for 12 to 24 hours.

Another object of the invention is to provide a multiparticulate modified release composition capable of releasing a cephalosporin in a bimodal or multi- modal manner in which a first portion of the active is released either immediately or after a delay time to provide a pulse of drug release and one or more additional portions of the active are released each after a respective lag time to provide additional pulses of drug release.

Another object of the invention is to provide solid oral dosage forms comprising a multiparticulate modified release composition of the present invention.

Other objects of the invention include provision of a once daily dosage form of a cephalosporin such as cefcapene pivoxil which, in operation, produces a plasma profile substantially similar to the plasma profile produced by the administration of two immediate release dosage forms given sequentially and a method for treatment of bacterial infection based on the administration of such a dosage form.

### DETAILED DESCRIPTION OF THE INVENTION

The above objects are realized by a multiparticulate modified release composition having a first component comprising a first population of cephalosporin particles, preferably cefcapene pivoxil and salts thereof and a second component comprising a second population of cephalosporin particles, preferably comprised of cefcapene pivoxil and salts thereof. The ingredient- containing particles of the second component are coated with a modified release coating. Alternatively or additionally, the second population of cephalosporin-containing particles further comprises a modified release matrix material. Following oral delivery, the composition in operation delivers the cephalosporin in a pulsatile manner.

In a preferred embodiment, the multiparticulate modified release composition of the present invention comprises a first component which is an immediate release component.

The modified release coating applied to the second population of cephalosporin particles causes a lag time between the release of active from the first population of active cephalosporin containing particles and the release of active from the second population of active cephalosporin-containing particles. Similarly, the presence of a modified release matrix material in the second population of active cephalosporin containing particles causes a lag time between the release of cephalosporin from the first population of cephalosporin-containing particles and the release of active ingredient from the second population of active ingredient containing particles. The duration of the lag time may be varied by altering the composition and/or the amount of the modified release coating and/or altering the composition and/or amount of modified release matrix material utilized. Thus, the duration of the lag time can be designed to mimic a desired plasma profile.

Because the plasma profile produced by the multiparticulate modified release composition upon administration is substantially similar to the plasma profile produced by the administration of two or more IR dosage forms given sequentially, the multiparticulate controlled release composition of the present invention is particularly useful for administering cephalosporin, particularly cefcapene pivoxil or a salt thereof for which patient tolerance may be problematical. This multiparticulate modified release composition is therefore advantageous for reducing or minimizing the development of patient tolerance to the active ingredient in the composition.

In a preferred embodiment of the present invention, the active cephalosporin is cefcapene pivoxil or a salt thereof and the composition in operation delivers the cefcapene pivoxil or salt thereof in a bimodal or pulsatile manner. Such a composition in operation produces a plasma profile which substantially mimics that obtained by the sequential administration of two IR doses as, for instance, in a typical antibiotic treatment regimen.

The present invention also provides solid oral dosage forms comprising a composition according to the invention.

The present invention further provides a method of treating a patient suffering from a bacterial infection utilizing a cephalosporin, preferably cefcapene pivoxil or a salt thereof, comprising the administration of a therapeutically effective amount of a solid oral dosage form of a cephalosporin to provide a pulsed or bimodal delivery of the cephalosporin, preferably cefcapene pivoxil or a salt thereof Advantages of the present invention include reducing the dosing frequency required by conventional multiple IR dosage regimes while still maintaining the benefits derived from a pulsatile plasma profile. This reduced dosing frequency is advantageous in terms of patient compliance to have a formulation which may be administered at reduced frequency. The reduction in dosage frequency made possible by utilizing the present invention would contribute to reducing health care costs by reducing the amount of time spent by health care workers on the administration of drugs.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixtures thereof, irrespective of their size, shape or morphology.

The term “modified release” as used herein with respect to the coating or coating material or used in any other context, means release which is not immediate release and is taken to encompass controlled release, sustained release and delayed release.

The term “time delay” as used herein refers to the duration of time between administration of the composition and the release of the cephalosporin, preferably cefcapene privoxil or a salt thereof, from a particular component.

The term “lag time” as used herein refers to the time between delivery of the cephalosporin from one component and the subsequent delivery of cephalosporin, preferably cefcapene privoxil or a salt thereof, from another component.

The term “erodable” as used herein refers to formulations which may be worn away, diminished, or deteriorated by the action of substances within the body.

The term “diffusion controlled” as used herein refers to formulations which may spread as the result of their spontaneous movement, for example, from a region of higher to one of lower concentration.

The term “osmotic controlled” as used herein refers to formulations which may spread as the result of their movement through a semipermeable membrane into a solution of higher concentration that tends to equalize the concentrations of the formulation on the two sides of the membrane.

The active ingredient in each component may be the same or different. For example, a composition may comprise a first component containing cefcapene pivoxil or a salt thereof, and the second component may comprise a second active ingredient which would be desirable for combination therapies. Indeed, two or more active ingredients may be incorporated into the same component when the active ingredients are compatible with each other. A drug compound present in one component of the composition may be accompanied by, for example, an enhancer compound or a sensitizer compound in another component of the composition, in order to modify the bioavailability or therapeutic effect of the drug compound.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

The proportion of the cephalosporin, preferably cefcapene pivoxil or a salt thereof, contained in each component may be the same or different depending on the desired dosing regime. The cephalosporin is present in the first component and in the second component in any amount sufficient to elicit a therapeutic response. The cephalosporin, when applicable, may be present either in the form of one substantially optically pure enantiomer or as a mixture, racemic or otherwise, of enantiomers. The cephalosporin is preferably present in a composition in an amount of from 0.1-500 mg, preferably in the amount of from 1-100 mg. Cephalosporin is preferably present in the first component in an amount of from 0.5-60 mg; more preferably the cephalosporin, is present in the first component in an amount of from 2.5-30 mg. The cephalosporin is present in the subsequent components in an amount within a similar range to that described for the first component.

The time release characteristics for the delivery of the cephalosporin, preferably cefcapene pivoxil or a salt thereof, from each of the components may be varied by modifying the composition of each component, including modifying any of the excipients or coatings which may be present. In particular, the release of cephalosporin may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time or delay time for the release of the cephalosporin, preferably cefcapene pivoxil or a salt thereof, from each component may also be varied by modifying the composition of each of the components, including modifying any excipients and coatings which may be present. For example, the first component may be an immediate release component wherein the cephalosporin is released immediately upon administration. Alternatively, the first component may be, for example, a time-delayed immediate release component in which the cephalosporin is released substantially in its' entirety immediately after a time delay. The second component may be, for example, a time-delayed immediate release component as just described or, alternatively, a time-delayed sustained release or extended release component in which the cephalosporin is released in a controlled fashion over an extended period of time.

As will be appreciated by those skilled in the art, the exact nature of the plasma concentration curve will be influenced by the combination of all of these factors just described. In particular, the lag time between the delivery (and thus also the on-set of action) of the cephalosporin in each component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of the cephalosporin from each component and the nature of the release of the antibiotic from each component (i.e. immediate release, sustained release etc.), the pulses in the plasma profile may be well separated and clearly defined peaks (e.g. when the lag time is long) or the pulses may be superimposed to a degree (e.g. in when the lag time is short).

In a preferred embodiment, the multi-particulate modified release composition according to the present invention has an immediate release component and at least one modified release component, the immediate release component comprising a first population of active ingredient containing particles and the modified release components comprising second and subsequent populations of active ingredient containing particles. The second and subsequent modified release components may comprise a controlled release coating. Additionally or alternatively, the second and subsequent modified release components may comprise a modified release matrix material. In operation, administration of such a multi-particulate modified release composition having, for example, a single modified release component results in characteristic pulsatile plasma concentration levels of the cephalosporin, preferably cefcapene pivoxil or a salt thereof, in which the immediate release component of the composition gives rise to a first peak in the plasma profile and the modified release component gives rise to a second peak in the plasma profile. Embodiments of the invention comprising more than one modified release component give rise to further peaks in the plasma profile.

Such a plasma profile produced from the administration of a single dosage unit is advantageous when it is desirable to deliver two (or more) pulses of active ingredient without the need for administration of two (or more) dosage units. Additionally, in the case of bacterial infection, it is particularly useful to have such a bimodal plasma profile. For example, a typical cefcapene pivoxil hydrochloride treatment regime consists of administration of three doses of an immediate release dosage formulation given four hours apart. This type of regime has been found to be therapeutically effective and is widely used. As previously mentioned, the development of patient tolerance is an adverse effect sometimes associated with cefcapene pivoxil HCl treatments. It is believed that the trough in the plasma profile between the two peak plasma concentrations is advantageous in reducing the development of patient tolerance by providing a period of wash out of the cefcapene pivoxil. Drug delivery systems which provide zero order or pseudo zero order delivery of the cefcapene pivoxil do not facilitate this wash out process.

Any coating material which modifies the release of the cephalosporin, preferably cefcapene pivoxil of a salt thereof, in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit.RTM. RS and RL, poly acrylic acid and poly acrylate and methacrylate copolymers such as those sold under the Trade Mark Eudragite S and L, polyvinyl acetaldiethylamino acetate, hydroxypropyl methylcellulose acetate succinate, shellac; hydrogels and gel-forming materials, such as carboxyvinyl polymers, sodium alginate, sodium cannellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit.RTM. RS-PM, Rohm & Haas), pullulan, collagen, casein, agar, gum arabic, sodium carboxymethyl cellulose, (swellable hydrophilic polymers) poly(hydroxyalkyl methacrylate) (m. wt. .about.5 k-5,000 k), polyvinylpyrrolidone (m. wt. .about.10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. .about.30 k-300 k), polysaccharides such as agar, acacia, karaya, tragacanth, algins and guar, polyacrylamides, Polyox.RTM. polyethylene oxides (m. wt. .about. 100 k-5,000 k), AquaKeep.RTM. acrylate polymers, diesters of polyglucan, crosslinked polyvinyl alcohol and poly N- vinyl-2-pyrrolidone, sodium starch glucolate (e.g. Explotab.RTM.; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox.RTM., Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit.RTM., Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of cephalosporin, preferably cefcapene pivoxil or a salt thereof, dispersed therein in vitro or in vivo. Modified release matrix materials suitable for the practice of the present invention include but are not limited to microcrytalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

A multiparticulate modified release composition according to the present invention may be incorporated into any suitable dosage form which facilitates release of the active ingredient in a pulsatile manner. Typically, the dosage form may be a blend of the different populations of cephalosporin- containing particles which make up the immediate release and the modified release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet. In this instance the first component of the multiparticulate modified release composition may be compressed into one layer, with the second component being subsequently added as a second layer of the multilayer tablet. The populations of cephalosporin-containing particles making up the composition of the invention may further be included in rapidly dissolving dosage forms such as an effervescent dosage form or a fast-melt dosage form.

The composition according to the invention comprises at least two populations of cephalosporin-containing particles which have different in vitro dissolution profiles.

Preferably, in operation the composition of the invention and the solid oral dosage forms containing the composition release the cephalosporin, preferably cefcapene pivoxil or a salt thereof such that substantially all of the cephalosporin contained in the first component is released prior to release of the cephalosporin from the second component. When the first component comprises an IR component, for example, it is preferable that release of the cephalosporin from the second component is delayed until substantially all the cephalosporin in the IR component has been released. Release of the cephalosporin from the second component may be delayed as detailed above by the use of a modified release coating and/or a modified release matrix material.

More preferably, when it is desirable to minimize patient tolerance by providing a dosage regime which facilitates wash-out of a first dose of cephalosporin, preferably cefcapene pivoxil or a salt thereof from a patient's system, release of the cephalosporin from the second component is delayed until substantially all of the cephalosporin contained in the first component has been released, and further delayed until at least a portion of the cephalosporin released from the first component has been cleared from the patient's system. In a preferred embodiment, release of the cephalosporin from the second component of the composition in operation is substantially, if not completely, delayed for a period of at least about two hours after administration of the composition.

The cephalosporin release of the drug from the second component of the composition in operation is substantially, if not completely, delayed for a period of at least about four hours, preferably about four hours, after administration of the composition.

As described herein, the invention includes various types of controlled release systems by which the active drug may be delivered in a pulsatile manner. These systems include, but are not limited to: films with the drug in a polymer matrix (monolithic devices); the drug contained by the polymer (reservoir devices); polymeric colloidal particles or microencapsulates (microparticles, microspheres or nanoparticles) in the form of reservoir and matrix devices; drug contained by a polymer containing a hydrophilic and/or leachable additive eg, a second polymer, surfactant or plasticiser, etc. to give a porous device, or a device in which the drug release may be osmotically ‘controlled’ (both reservoir and matrix devices); enteric coatings (ionise and dissolve at a suitable pH); (soluble) polymers with (covalently) attached ‘pendant’ drug molecules; devices where release rate is controlled dynamically: eg, the osmotic pump.

The delivery mechanism of the invention will control the rate of release of the drug. While some mechanisms will release the drug at a constant rate (zero order), others will vary as a function of time depending on factors such as changing concentration gradients or additive leaching leading to porosity, etc.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Röhm Pharma, Weiterstadt.) range of poly(acrylate, methacrylate) copolymers are known in the art.

### Reservoir Devices

A typical approach to controlled release is to encapsulate or contain the drug entirely (eg, as a core), within a polymer film or coat (ie, microcapsules or spray/pan coated cores).

The various factors that can affect the diffusion process may readily be applied to reservoir devices (eg, the effects of additives, polymer functionality {and, hence, sink-solution pH} porosity, film casting conditions, etc.) and, hence, the choice of polymer must be an important consideration in the development of reservoir devices. Modelling the release characteristics of reservoir devices (and monolithic devices) in which the transport of the drug is by a solution-diffusion mechanism therefore typically involves a solution to Fick's second law (unsteady-state conditions; concentration dependent flux) for the relevant boundary conditions. When the device contains dissolved active agent, the rate of release decreases exponentially with time as the concentration (activity) of the agent (ie, the driving force for release) within the device decreases (ie, first order release). If, however, the active agent is in a saturated suspension, then the driving force for release is kept constant (zero order) until the device is no longer saturated. Alternatively the release-rate kinetics may be desorption controlled, and a function of the square root of time.

Transport properties of coated tablets, may be enhanced compared to free-polymer films, due to the enclosed nature of the tablet core (permeant) which may enable the internal build-up of an osmotic pressure which will then act to force the permeant out of the tablet.

The effect of deionised water on salt containing tablets coated in poly(ethylene glycol) (PEG)-containing silicone elastomer, and also the effects of water on free films has been investigated. The release of salt from the tablets was found to be a mixture of diffusion through water filled pores, formed by hydration of the coating, and osmotic pumping. KCI transport through films containing just 10% PEG was negligible, despite extensive swelling observed in similar free films, indicating that porosity was necessary for the release of the KCl which then occurred by ‘trans-pore diffusion.’ Coated salt tablets, shaped as disks, were found to swell in deionised water and change shape to an oblate spheroid as a result of the build-up of internal hydrostatic pressure: the change in shape providing a means to measure the ‘force’ generated. As might be expected, the osmotic force decreased with increasing levels of PEG content. The lower PEG levels allowed water to be imbibed through the hydrated polymer; whilst the porosity resulting from the coating dissolving at higher levels of PEG content (20 to 40%) allowed the pressure to be relieved by the flow of KCl.

Methods and equations have been developed, which by monitoring (independently) the release of two different salts (eg, KCl and NaCl) allowed the calculation of the relative magnitudes that both osmotic pumping and trans-pore diffusion contributed to the release of salt from the tablet. At low PEG levels, osmotic flow was increased to a greater extent than was trans-pore diffusion due to the generation of only a low pore number density: at a loading of 20%, both mechanisms contributed approximately equally to the release. The build-up of hydrostatic pressure, however, decreased the osmotic inflow, and osmotic pumping. At higher loadings of PEG, the hydrated film was more porous and less resistant to outflow of salt. Hence, although the osmotic pumping increased (compared to the lower loading), trans-pore diffusion was the dominant release mechanism. An osmotic release mechanism has also been reported for microcapsules containing a water soluble core.

### Monolithic Devices (Matrix Devices)

Monolithic (matrix) devices are possibly the most common of the devices for controlling the release of drugs. This is possibly because they are relatively easy to fabricate, compared to reservoir devices, and there is not the danger of an accidental high dosage that could result from the rupture of the membrane of a reservoir device. In such a device the active agent is present as a dispersion within the polymer matrix, and they are typically formed by the compression of a polymer/drug mixture or by dissolution or melting. The dosage release properties of monolithic devices may be dependent upon the solubility of the drug in the polymer matrix or, in the case of porous matrixes, the solubility in the sink solution within the particle's pore network, and also the tortuosity of the network (to a greater extent than the permeability of the film), dependent on whether the drug is dispersed in the polymer or dissolved in the polymer. For low loadings of drug, (0 to 5% W/V) the drug will be released by a solution-diffusion mechanism (in the absence of pores). At higher loadings (5 to 10% WNV), the release mechanism will be complicated by the presence of cavities formed near the surface of the device as the drug is lost: such cavities fill with fluid from the environment increasing the rate of release of the drug.

It is common to add a plasticiser (eg, a poly(ethylene glycol)), or surfactant, or adjuvant (ie, an ingredient which increases effectiveness), to matrix devices (and reservoir devices) as a means to enhance the permeability (although, in contrast, plasticiser may be fugitive, and simply serve to aid film formation and, hence, decrease permeability—a property normally more desirable in polymer paint coatings). It was noted that the leaching of PEG acted to increase the permeability of (ethyl cellulose) films linearly as a function of PEG loading by increasing the porosity, however, the films retained their barrier properties, not permitting the transport of electrolyte. It was deduced that the enhancement of their permeability was as a result of the effective decrease in thickness caused by the PEG leaching. This was evinced from plots of the cumulative permeant flux per unit area as a function of time and film reciprocal thickness at a PEG loading of 50% W/W: plots showing a linear relationship between the rate of permeation and reciprocal film thickness, as expected for a (Fickian) solution-diffusion type transport mechanism in a homogeneous membrane. Extrapolation of the linear regions of the graphs to the time axis gave positive intercepts on the time axis: the magnitude of which decreased towards zero with decreasing film thickness. These changing lag times were attributed to the occurrence of two diffusional flows during the early stages of the experiment (the flow of the ‘drug’ and also the flow of the PEG), and also to the more usual lag time during which the concentration of permeant in the film is building-up. Caffeine, when used as a permeant, showed negative lag times. No explanation of this was forthcoming, but it was noted that caffeine exhibited a low partition coefficient in the system, and that this was also a feature of aniline permeation through polyethylene films which showed a similar negative time lag.

The effects of added surfactants on (hydrophobic) matrix devices has been investigated. It was thought that surfactant may increase the drug release rate by three possible mechanisms: (i) increased solubilisation, (ii) improved ‘wettability’ to the dissolution media, and (iii) pore formation as a result of surfactant leaching. For the system studied (Eudragit®RL 100 and RS 100 plasticised by sorbitol, Flurbiprofen as the drug, and a range of surfactants) it was concluded that improved wetting of the tablet led to only a partial improvement in drug release (implying that the release was diffusion, rather than dissolution, controlled), although the effect was greater for Eudragit® RS than Eudragit® RL, whilst the greatest influence on release was by those surfactants that were more soluble due to the formation of ‘disruptions’ in the matrix allowing the dissolution medium access to within the matrix. This is of obvious relevance to a study of latex films which might be suitable for pharmaceutical coatings, due to the ease with which a polymer latex may be prepared with surfactant as opposed to surfactant-free. Differences were found between the two polymers—with only the Eudragit® RS showing interactions between the anionic/cationic surfactant and drug. This was ascribed to the differing levels of quaternary ammonium ions on the polymer.

Composite devices consisting of a polymer/drug matrix coated in a polymer containing no drug also exist. Such a device was constructed from aqueous Eudragit latices, and was found to give zero order release by diffusion of the drug from the core through the shell. Similarly, a polymer core containing the drug has been produced, but coated this with a shell that was eroded by the gastric fluid. The rate of release of the drug was found to be relatively linear (a function of the rate limiting diffusion process through the shell) and inversely proportional to the shell thickness, whereas the release from the core alone was found to decrease with time.

### Microspheres

Methods for the preparation of hollow microspheres (‘microballoons’) with the drug dispersed in the sphere's shell, and also highly porous matrix-type microspheres (‘microsponges’) have been described. The microsponges were prepared by dissolving the drug and polymer in ethanol. On addition to water, the ethanol diffused from the emulsion droplets to leave a highly porous particle.

The hollow microspheres were formed by preparing a solution of ethanol/dichloro-methane containing the drug and polymer. On pouring into water, this formed an emulsion containing the dispersed polymer/drug/solvent particles, by a coacervation-type process, from which the ethanol (a good solvent for the polymer) rapidly diffused precipitating polymer at the surface of the droplet to give a hard-shelled particle enclosing the drug, dissolved in the dichloromethane. At this point, a gas phase of dichloromethane was generated within the particle which, after diffusing through the shell, was observed to bubble to the surface of the aqueous phase. The hollow sphere, at reduced pressure, then filled with water, which could be removed by a period of drying. (No drug was found in the water.) A suggested use of the microspheres was as floating drug delivery devices for use in the stomach.

### Pendent Devices

A means of attaching a range of drugs such as analgesics and antidepressants, etc., by means of an ester linkage to poly(acrylate) ester latex particles prepared by aqueous emulsion polymerization has been developed. These latices when passed through an ion exchange resin such that the polymer end groups were converted to their strong acid form could ‘self-catalyse’ the release of the drug by hydrolysis of the ester link.

Drugs have been attached to polymers, and also monomers have been synthesized with a pendent drug attached. The research group have also prepared their own dosage forms in which the drug is bound to a biocompatible polymer by a labile chemical bond eg, polyanhydrides prepared from a substituted anhydride (itself prepared by reacting an acid chloride with the drug: methacryloyl chloride and the sodium salt of methoxy benzoic acid) were used to form a matrix with a second polymer (Eudragite RL) which released the drug on hydrolysis in gastric fluid. The use of polymeric Schiffbases suitable for use as carriers of pharmaceutical amines has also been described.

Enteric Films Enteric coatings consist of pH sensitive polymers. Typically the polymers are carboxylated and interact (swell) very little with water at low pH, whilst at high pH the polymers ionise causing swelling, or dissolving of the polymer. Coatings can therefore be designed to remain intact in the acidic environment of the stomach (protecting either the drug from this environment or the stomach from the drug), but to dissolve in the more alkaline environment of the intestine.

### Osmotically Controlled Devices

The osmotic pump is similar to a reservoir device but contains an osmotic agent (eg, the active agent in salt form) which acts to imbibe water from the surrounding medium via a semi-permeable membrane. Such a device, called the ‘elementary osmotic pump’, has been described. Pressure is generated within the device which forces the active agent out of the device via an orifice (of a size designed to minimise solute diffusion, whilst preventing the build-up of a hydrostatic pressure head which has the effect of decreasing the osmotic pressure and changing the dimensions {volume} of the device). Whilst the internal volume of the device remains constant, and there is an excess of solid (saturated solution) in the device, then the release rate remains constant delivering a volume equal to the volume of solvent uptake.

### Electrically Stimulated Release Devices

Monolithic devices have been prepared using polyelectrolyte gels which swelled when, for example, an external electrical stimulus was applied, causing a change in pH. The release could be modulated, by the current, giving a pulsatile release profile.

### Hydrogels

Hydrogels find a use in a number of biomedical applications, in addition to their use in drug matrices (eg, soft contact lenses, and various ‘soft’ implants, etc.)

In the following examples all percentages are weight by weight unless otherwise stated. The term “purified water” as used throughout the Examples refers to water that has been purified by passing it through a water filtration system. It is to be understood that the examples are for illustrative purposes only, and should not be interpreted as restricting the spirit and scope of the invention, as defined by the scope of the claims that follow.

### EXAMPLE 1

### Multiparticulate Modified Release Composition Containing Cefcapene Pivoxil HCl

A multiparticulate modified release composition according to the present invention comprising an immediate release component and a modified release component containing cefcapene pivoxil HCl is prepared as follows.

(a) Immediate Release Component.

A solution of cefcapene pivoxil HCl (50:50 racemic mixture) is prepared according to any of the formulations given in Table 1. The methylphenidate solution is then coated onto nonpareil seeds to a level of approximately 16.9% solids weight gain using, for example, a Glatt GPCG3 (Glatt, Protech Ltd., Leicester, UK) fluid bed coating apparatus to form the IR particles of the immediate release component.

TABLE 1
Immediate release component solutions

| | Amount, | % (w/w) |
|---|---|---|
| Ingredient | (i) | (ii) |
| Cefcapene Pivoxil HCl | 13.0 | 13.0 |
| Polyethylene Glycol 6000 | 0.5 | 0.5 |
| Polyvinylpyrrolidone | 3.5 | |
| Purified Water | 83.5 | 86.5 |

(b) Modified Release Component

Cefcapene pivoxil HCl containing delayed release particles are prepared by coating immediate release particles prepared according to Example 1 (a) above with a modified release coating solution as detailed in Table 2. The immediate release particles are coated to varying levels up to approximately to 30% weight gain using, for example, a fluid bed apparatus.

TABLE 2
Modified release component coating solutions

| | Amount, % (w/w) | | | | | | | |
|---|---|---|---|---|---|---|---|---|
| Ingredient | (i) | (ii) | (iii) | (iv) | (v) | (vi) | (vii) | (viii) |
| Eudragit ® | 49.7 | 42.0 | 47.1 | 53.2 | 40.6 | — | — | 25.0 |
| RS 12.5 | | | | | | | | |
| Eudragit ® | — | — | — | — | — | 54.35 | 46.5 | — |
| S 12.5 | | | | | | | | |
| Eudragit ® | — | — | — | — | — | — | 25.0 | |
| L 12.5 | | | | | | | | |
| Polyvinyl- | — | — | — | 0.35 | 0.3 | — | — | |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethyl- | — | — | — | — | — | — | — | 1.25 |
| citrate | | | | | | | | |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.4 | — | — | — |
| Talc¹ | — | 16.0 | 5.9 | — | 16.3 | — | 2.8 | 2.25 |

¹Talc is simultaneously applied during coating for formulations in column (i), (iv) and (vi).

(c) Encapsulation of Immediate and Delayed Release Particles.

The immediate and delayed release particles prepared according to Example 1 (a) and (b) above are encapsulated in size 2 hard gelatin capsules to an overall 20 mg dosage strength using, for example, a Bosch GKF 4000S encapsulation apparatus. The overall dosage strength of 20 mg cefcapene pivoxil HCl was made up of 10 mg from the immediate release component and 10 mg from the modified release component.

### EXAMPLE 2

### Multiparticulate Modified Release Composition Containing Cefcapene Pivoxil HCl

Multiparticulate modified release cefcapene pivoxil HCl compositions according to the present invention having an immediate release component and a modified release component having a modified release matrix material are prepared according to the formulations shown in Table 5(a) and (b).

TABLE 5(a)
100 mg of IR component is encapsulated with 100 mg of modified
release (MR) component to give a 20 mg dosage strength product

| | % (w/w) |
|---|---|
| IR component | |
| Cefcapene Pivoxil HCl | 10 |
| Microcrytalline cellulose | 40 |
| Lactose | 45 |
| Povidone | 5 |
| MR component | |
| Cefcapene Pivoxil HCl | 10 |
| Microcrytalline cellulose | 40 |
| Eudragit .RTM. RS | 45 |
| Povidone | 5 |

TABLE 5(b)
50 mg of IR component is encapsulated with 50 mg of modified
release (MR) component to give a 20 mg dosage strength product.

| | % (w/w) |
|---|---|
| IR component | |
| Cefcapene Pivoxil HCl | 20 |
| Microcrystalline cellulose | 50 |
| Lactose | 28 |
| Povidone | 2 |
| MR component | |
| Cefcapene Pivoxil HCl | 20 |
| Microcrytalline cellulose | 50 |
| Eudragit ® S | 28 |
| Povidone | 2 |

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

1. A controlled release antibiotic composition comprising a first population of cephalosporin-containing particles and at least one subsequent population of cephalosporin-containing particles, wherein the cephalosporin contained in the first population is substantially uncoated, and the subsequent population of cephalosporin-containing particles further comprises a modified release coating or, alternatively or additionally, a modified release matrix material, such that the composition following oral delivery to a subject delivers the cephalosporin in the first and subsequent populations in a pulsatile manner.

2. The controlled release composition of claim 1, wherein said cephalosporin is cefcapene pivoxil or a salt thereof.

3. The composition according to claim 2, wherein the first population comprises immediate-release particles and the subsequent population comprises modified-release particles.

4. The composition according to claim 2, wherein the first population comprises immediate-release particles and the formulation comprising the subsequent population is an erodable formulation.

5. The composition according to claim 2, wherein the formulation comprising the subsequent population is a diffusion controlled formulation.

6. The composition according to claim 2, wherein the formulation comprising the subsequent population is an osmotic controlled formulation.

7. The composition according to claim 3, wherein the modified release particles have a modified-release coating.

8. The composition according to claim 3, wherein the modified release particles comprise a modified-release matrix material.

9. The compositions of claim 7 or 8 wherein said modified release particles are combined in formulation that releases said cefcapene pivoxil or salt thereof by erosion, diffusion or osmosis to the surrounding environment.

10. The composition according to claim 9, wherein at least one of the first and subsequent populations further comprises an enhancer.

11. The composition according to claim 10, wherein the amount of active ingredient contained in each of the first and subsequent populations is from about 0.1 mg to about 1 g.

12. The composition according to claim 11, wherein the first and subsequent populations have different in vitro dissolution profiles.

13. The composition according to claim 12, which in operation releases substantially all of the cefcapene pivoxil from the first population prior to release of the antibiotic from the subsequent population.

14. The dosage form according to claim 13 comprising a blend of the particles of each of the first and subsequent populations contained in a hard gelatin or soft gelatin capsule.

15. The dosage form according to claim 14, wherein the particles of each of the populations are in the form of mini-tablets and the capsule contains a mixture of the mini-tablets.

16. The dosage form according to claim 13, in the form of a multilayer tablet comprising a first layer of compressed cefapene pivoxil or salt thereof-containing particles of the first population and another layer of compressed antibiotic-containing particles of the subsequent population.

17. The dosage form according to claim 16, wherein the first and subsequent populations of cefcapene pivoxil or salt thereof-containing particles are provided in a rapidly dissolving dosage form.

18. The dosage form according to claim 17, comprising a fast-melt tablet.

19. A method for the treatment of bacterial infection comprising administering a therapeutically effective amount of a composition according to claim 2.

20. The composition according to claim 2, wherein the modified-release particles comprise a pH-dependent polymer coating which is effective in releasing a pulse of the active ingredient following a time delay.

21. The composition according to claim 20, wherein the polymer coating comprises methacrylate copolymers.

22. The composition according to claim 21, wherein the polymer coating comprises a mixture of methacrylate and ammonio methacrylate copolymers in a ratio sufficient to achieve a pulse of the active ingredient following a time delay.

23. The composition according to claim 22, wherein the ratio of methacrylate to ammonio methacrylate copolymers is 1:1.
